(19) United States
(12) Patent Application Publication
(10) Pub. No.: US 2024/0424066 A1
(43) Pub. Date: Dec. 26, 2024

(54) Chondroprotective Nutraceutical Composition and Method of Using Same

(71) Applicant: Lonza Greenwood LLC, Greenwood, SC (US)

(72) Inventors: Vijaya Juturu, North Bruswick, NJ (US); Zainulabedin Saiyed, Benicia, CA (US); Shane Durkee, Morristown, NJ (US)

(21) Appl. No.: 18/701,644

(22) PCT Filed: Oct. 19, 2022
(86) PCT No.: PCT/US2022/047076

§ 371 Date: Apr. 16, 2024

Related U.S. Application Data
(60) Provisional application No. 63/257,170, filed on Oct. 19, 2021.

(51) Int. Cl. A61K 38/39; A23L 33/17; A61K 33/06; A61P 3/14; A61P 19/08

(52) U.S. Cl. CPC A61K 38/39; A23L 33/17; A61K 33/06; A61P 3/14; A61P 19/08

(57) ABSTRACT

A chondroprotective nutraceutical composition and method are disclosed capable of increasing calcium absorption and retention in the skeletal structure of a mammal. The chondroprotective nutraceutical composition contains a collagen. In addition to improving calcium intake, the chondroprotective nutraceutical composition of the present disclosure can also improve various other bone health markers. The composition can be used in the form of a supplement for preventing bone disease and/or treating bone ailments.

### BACKGROUND

Skeletal bones in mammals provide support, enable walking, sitting and bending, and also protect the brain and the various organs contained in the body. Poor bone health can affect all different types of mammals, including canines, livestock, and humans. Poor bone health, however, can be difficult to detect in that individuals are typically unaware of a problem until stress is applied to the bones that causes a fracture or break. Especially as mammals age, bones may start to wear down faster than the body can repair them resulting in a frail bone structure that can occur without any perceived side effects until the condition results in an injury.

Humans, for instance, can lose up to 2% of their bone density every year after reaching age 50. Loss of bone density can result in osteoporosis. Osteoporosis is a disabling disease characterized by the loss of bone mass and microarchitectural deterioration of the skeletal structure leading to compromised bone strength, which predisposes a mammal, such as a human, to increased risk of fragility fractures. Osteoporosis affects more than 70 million people in the United States, Europe and Japan alone, leading to millions of fractures in patients each year. Osteoporosis can affect greater than 25% of all post-menopausal women that increases as the population grows older. For example, greater than 30% of all women older than 50 years old will have an osteoporotic fracture. Older men can also be similarly affected by osteoporosis, leading many times to hip fractures.

Osteoporosis can be caused by various different factors, including metabolic changes in the body in combination with a diet that is low in bone health nutrients. Osteoporosis can also occur as a result of medications or as a side effect to contracting a disease. In fact, the long-term effects on bone health due to the Covid pandemic are still not known.

Throughout life, bone is continuously remodeled with resorption of old bone (catabolic process) performed by osteoclasts and deposition of new bone (anabolic process) performed by osteoblasts. Bone remodeling is not a random process and takes place in focal bone multicellular units (BMUs), which are remodeling units comprising osteoblasts, osteoclasts, and their precursors, in which resorption and formation are coupled. Bone resorption is likely the initial event that occurs in response to local mechanical stress signals. The reduction in bone density found in osteoporosis results from an imbalance between resorption and formation, wherein the rate of resorption exceeds that of formation. Osteoporosis represents a continuum, in which multiple pathogenetic mechanisms converge to cause loss of bone mass and microarchitectural deterioration of skeletal structure. Osteoporosis is likely to be caused by complex interactions among local and systemic regulators of bone cell function. The heterogeneity of osteoporosis may be due not only to differences in the production of systemic and local regulators, but also to changes in receptors, signal transduction mechanisms, nuclear transcription factors, and enzymes that produce or inactivate local regulators.

Bone strength reflects the integration of two main features: bone density and bone quality. Bone density is expressed as grams of mineral per area or volume and, in any given individual, is determined by peak bone mass attained and subsequent amount of bone loss. Bone quality refers to architecture, turnover, damage accumulation (i.e., microfractures) and mineralization. A fracture frequently occurs when trauma is applied to osteoporotic bone, which is of a lower bone density. Thus, osteoporosis is a significant risk factor for bone fractures.

As indicated above, diagnosing osteoporosis can be difficult. Typically, osteoporosis is detected by a bone mineral density test. A majority of affected patients, however, are not aware that they have a low bone density or at risk until after some type of bone fracture occurs.

In view of the above, there remains a need in the art for a method and supplement for preventing the onset of a bone disease, such as osteoporosis. In another aspect, a need also exists for a method and supplement that can improve bone and joint health including the health of tendons and ligaments associated with the skeletal system of a mammal.

### SUMMARY

In general, the present disclosure is directed to a chondroprotective nutraceutical composition that is capable of increasing the absorption of at least one bone health additive into the skeletal system of a mammal including associated tendons and ligaments. The mammal is not limited and can be a human, a canine, a feline, a bovine, a horse or the like. The present disclosure is also directed to a method for increasing the absorption of at least nutrient, such as a bone health additive, into the bones, tendons, and ligaments of a mammal by administering to the mammal a therapeutically and/or nutritionally effective amount of a chondroprotective nutraceutical composition. The bone health additive can be already present in the diet of the mammal and/or can be added in conjunction with the chondroprotective nutraceutical composition. In accordance with the present disclosure, the chondroprotective nutraceutical composition contains at least one collagen source.

For example, in one embodiment, the present disclosure is directed to a method for increasing calcium absorption in a mammal. The mammal can have a diet that includes calcium. The method comprises administering to the mammal a therapeutically effective amount of a chondroprotective nutraceutical composition sufficient to increase calcium absorption in the mammal. The chondroprotective nutraceutical composition can comprise a Type II collagen.

The chondroprotective nutraceutical composition can be administered to the mammal in order to increase or improve various bone health parameters. For example, the chondroprotective nutraceutical composition can be administered to the mammal in an amount sufficient to increase percent absorptivity and/or percent retentivity of the calcium contained in the diet of the mammal. In addition to calcium, the chondroprotective nutraceutical composition can also increase intake of various nutrients including vitamin D, vitamin K2, magnesium, herbals, and the like. The chondroprotective nutraceutical composition can also be administered to the mammal in an amount sufficient to increase bone weight, such as dry femur weight. The chondroprotective nutraceutical composition can also be administered to the mammal in an amount sufficient to increase various bone health markers, such as an increase in osteocalcin. In another aspect, the chondroprotective nutraceutical composition can be administered to the mammal in an amount sufficient to increase bone morphogenetic protein 2.

The composition can contain any suitable collagen source, including Type 1, Type II or Type III collagen. In one embodiment, the collagen can comprise undenatured Type II collagen. The collagen can be obtained from chicken cartilage, such as chicken sternum cartilage.

The chondroprotective nutraceutical composition can be administered to the mammal at least once a week, such as at least every three days. In one embodiment, the chondroprotective nutraceutical composition can be administered to the mammal daily. The chondroprotective nutraceutical composition can contain a collagen, such as Type II collagen, at a dose level of from about 10 mg to about 3,000 mg of collagen. Any suitable delivery form can be used, including capsules, tablets, a suspension, or the like.

In one embodiment, the method of the present disclosure further includes the step of administering to the mammal a calcium supplement. The calcium supplement, for instance, can be contained within the chondroprotective nutraceutical composition or can be administered to the mammal separately.

In one embodiment, the method is administered to a mammal whose diet is deficient in calcium.

The present disclosure is also directed to a chondroprotective supplement for improving bone health in a mammal including improving associated cartilage, tendons and ligaments. The chondroprotective supplement comprises a bone health additive in combination with a bone health adjuvant. The bone health adjuvant comprises a Type II collagen, such as an undenatured Type II collagen. The bone health adjuvant is present in the supplement in a therapeutically effective amount sufficient to increase absorption of the bone health additive into the skeletal structure or bones of the mammal.

In one embodiment, the bone health additive comprises calcium. Calcium can be present in the chondroprotective supplement, for example, in an amount from about 250 mg to about 2,500 mg. The calcium can be in any suitable form, such as calcium carbonate, calcium citrate, calcium gluconate, calcium lactate, calcium hydroxyapatite, or mixtures thereof.

Collagen can be present in the chondroprotective supplement in an amount greater than about 20 mg, such as in an amount greater than about 30 mg, such as in an amount greater than about 40 mg, such as in an amount greater than about 80 mg, such as in an amount greater than about 100 mg and in an amount less than about 1000 mg, such as in an amount less than about 350 mg.

Other features and aspects of the present disclosure are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a graph illustrating some of the results obtained in the example described below;

FIG. 2 is a graph illustrating some of the results obtained in the example described below;

FIG. 3 is a graph illustrating some of the results obtained in the example described below;

FIG. 4 is a graph illustrating some of the results obtained in the example described below;

FIG. 5 is a graph illustrating some of the results obtained in the example described below;

FIG. 6 is a graph illustrating some of the results obtained in the example described below;

FIG. 7 is a graph illustrating some of the results obtained in the example described below;

FIG. 8 is a graph illustrating some of the results obtained in the example described below;

FIG. 9 is a graph illustrating some of the results obtained in the example described below;

FIG. 10 is a graph illustrating some of the results obtained in the example described below;

FIG. 11 is a graph illustrating some of the results obtained in the example described below;

FIG. 12 is a graph illustrating some of the results obtained in the example described below;

FIG. 13 is a graph illustrating some of the results obtained in the example described below;

FIG. 14 is a graph illustrating some of the results obtained in the example described below.

Repeat use of reference characters in the present specification and drawing is intended to represent same or analogous features or elements of the invention.

### Definitions

As used herein, the terms “about,” “approximately,” or “generally,” when used to modify a value, indicates that the value can be raised or lowered by 10%, in one aspect, such as 8%, such as 5%, such as 4%, such as 3%, such as 2%, such as 1% in one aspect, and remain within the disclosed aspect.

The term “therapeutically effective amount” and/or “nutraceutically effective amount” as used herein, shall mean that dosage, or amount of a composition, that provides the specific pharmacological or nutritional response for which the composition is administered or delivered to mammals in need of such treatment. It is emphasized that the “effective amount”, administered to a particular subject in a particular instance, will not always be effective in treating the ailments or otherwise improve health as described herein, even though such dosage is deemed an “effective amount” by those skilled in the art. Specific subjects may, in fact, be “refractory” to an “effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “supplement” means a product in addition to the normal diet of the mammal but may be combined with a mammal's normal food or drink composition. The supplement may be in any form but not limited to a solid, liquid, gel, capsule, tablet or powder. A supplement may also be administered simultaneously with or as a component of a food composition which may comprise a food product, a beverage, a pet food, a snack, or a treat. In one embodiment, the beverage may be an activity drink.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation canine, equine, feline, bovine, ovine, human, or porcine mammals.

As used herein, “healthy” refers to the absence of illness or injury.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII).

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a chondroprotective nutraceutical composition for improving bone health, which includes improving the health of cartilage, tendons and ligaments associated with the skeletal system. The present disclosure is also directed to a method of administering the chondroprotective nutraceutical composition to mammals. In one aspect, the chondroprotective nutraceutical composition can be administered to mammals orally in the form of a supplement. The chondroprotective nutraceutical composition is administered to mammals in an amount sufficient to, in one embodiment, increase the absorption of one or more bone health additives into the skeletal system of the mammal. When administered to a mammal in a therapeutically effective amount, for example, the mammal may experience an increase in body weight, an increase in retention of one or more bone health additives, such as calcium, an increase in dry bone weight, such as dry femur weight, or the like. The chondroprotective nutraceutical composition of the present disclosure can also increase bone mineral density, increase bone mineral content, and/or increase the amount of calcium in the bones.

The chondroprotective nutraceutical composition of the present disclosure can be administered to healthy mammals and to mammals that may have an underlying injury or ailment of their skeletal system. Overall, the chondroprotective nutraceutical composition can enhance absorption and retention of one or more bone health additives. The composition may also enhance activity of the bioactive or bone health additive. The chondroprotective nutraceutical composition alone and or in combination with any mineral or vitamin can enhance all these outcomes in healthy and diseased mammalian populations. Conditions that can be treated or prevented according to the present disclosure include osteoporosis, any other bone disease, joint disease, inflammatory disease condition in bone and joints, co-morbidity conditions like diabetes, cardiometabolic syndrome and or any other chronic disease.

The chondroprotective nutraceutical composition of the present disclosure, when administered in a therapeutically effective amount to a mammal, can also affect in a positive way various different bone markers. For example, the mammal can experience an increase in osteocalcin and a decrease in parathyroid hormone. The mammal may also experience an increase in osteoprotegerin, an increase in RANK which promotes bone formation, and a decrease in RANK-L which regulates bone resorption. The mammal can also experience a decrease in SOX-9 for chondrogenic differentiation, which is expressed in chondrocytes. The mammal can also experience an increase in IGF-1 which is correlated with bone mineral density and an increase in bone morphogenic protein 2, which plays a role in osteoblast differentiation.

In accordance with the present disclosure, the chondroprotective nutraceutical composition contains collagen alone or in combination with other bone health additives. Although collagen has been found to improve joint health at various concentrations, it was completely unexpected that collagen can have a dramatic and synergistic impact on improving bone health when combined with one or more bone health additives. The one or more bone health additives can be present in the composition, can be present in the diet of the mammal, or can be present in both.

The chondroprotective nutraceutical composition of the present disclosure can be used to prevent or treat various different bone diseases or conditions. For example, the chondroprotective nutraceutical composition of the present disclosure is particularly well suited to being administered to mammals, who are predisposed or at risk for developing osteoporosis, osteopenia, bone fractures, including work animals, horses, humans such as athletes, and the like. The chondroprotective nutraceutical composition of the present disclosure is also well suited to treating mammals with any of the above-described bone ailments. For instance, the composition can be used to slow or stop the progression of osteoporosis or osteopenia or substantially delay or prevent their onset.

The chondroprotective nutraceutical composition of the present disclosure generally contains a collagen source that contains collagen alone or in combination with one or more bone health additives. In one embodiment, for instance, the chondroprotective nutraceutical composition of the present disclosure does not contain any other bone health additives. Instead, the collagen contained in the composition synergistically combines with bone health additives contained in the mammal's diet, such as calcium, in order to increase absorption of the bone health additive into the skeletal system of the mammal. In this manner, the chondroprotective nutraceutical composition of the present disclosure can enhance the activity of various additives already contained within the mammal's diet. For example, the mammal may have a diet that is deficient in one or more bone health additives, such as calcium. The chondroprotective nutraceutical composition of the present disclosure can optimize and maximize absorption of the bone health additive into the mammal such that an additional quantity of the additive is not needed in the mammal's diet.

Alternatively, the chondroprotective nutraceutical composition of the present disclosure may contain collagen in combination with one or more bone health additives. As described above, one bone health additive that may be incorporated into the composition can be an additive containing calcium. The calcium additive, for instance, can be any suitable calcium salt, such as calcium carbonate, calcium citrate, calcium gluconate, calcium lactate, calcium hydroxyapatite, or mixtures thereof.

Other bone health additives that may be present in the composition include a vitamin, such as Vitamin D3 and/or Vitamin K2, and one or more minerals alone or in combination with one or more vitamins. In one aspect, the bone health composition can be a plant extract comprising cissus quadrangularis.

In other embodiments, the collagen can be combined with an antiresorptive agent, an anabolic agent, a nonsteroidal anti-inflammatory drug (NSAID), any drug for treating osteoarthritis, or combinations thereof. Such agents include bisphosphonates, denosumab, estrogens, calcitonin, romosozumab, teriparatide, or combinations thereof. Biophosphonates that can be present include alendronate, ibandronate, risedronate, zoledronic acid, or combinations thereof.

The chondroprotective nutraceutical composition of the present disclosure can also contain various other minerals and nutrients, such as a magnesium source, a manganese source, or the like.

Once the chondroprotective nutraceutical composition is administered to a mammal in a therapeutically effective amount, the bone health of the mammal can be dramatically and unexpectedly improved by synergistically combining with bone health additives present in the mammal. Of particular advantage, the bone mineral density of the mammal can be increased. For example, after a period of time, such as 12 weeks, the bone mineral density of the mammal can increase by at least about 4%, such as at least 8%, such as at least 12%, such as at least 18%, such as at least 22%, such as at least 26%, such as at least 31%, such as at least 35%, and generally less than about 100%, such as less than about 80% (mg/cm²). Similarly, the bone mineral content of the mammal can be increased by at least about 8%, such as at least by about 15%, such as at least by about 20%, such as at least by about 25%, such as at least by about 30%, such as at least by about 35%, such as at least by about 40%, such as at least by about 45%, and generally less than about 110%. Osteocalcin can be increased by greater than about 10%, such as greater than about 15%, such as greater than about 25%, such as greater than about 35%, such as greater than about 45%, such as greater than about 55%, such as greater than about 65%, such as greater than about 75%, such as greater than about 85%, and generally less than about 200% over a similar period of time, such as 12 weeks. Parathyroid hormone (PTH), on the other hand, can decrease during administration of the chondroprotective nutraceutical composition. PTH, for instance, can decrease by greater than about 5%, such as greater than about 15%, such as greater than about 25%, such as greater than about 35%, such as greater than about 45%. The above percentages can be in comparison to the mammal prior to receiving the treatment or can be compared to the same mammal or a group of the same mammals that have not been administered the chondroprotective nutraceutical composition.

Mammals administered the chondroprotective nutraceutical composition of the present disclosure have been found to show a significant increase in calcium absorptivity and retentivity when the collagen source is administered to the mammal in combination with calcium, whether the calcium is part of the mammal's regular diet, contained within the composition administered to the mammal, or both. The absorption of minerals, such as calcium, is calculated by the following formula:

$$Absorption ( mg / day or μg / day ) = intake - fecal excretion$$

$$Absorptivity ( \% ) = absorption / intake \times 100$$

The retention of a mineral is calculated as follows:

$$Retention ( mg / day or μg / day ) = absorption - urinary excretion$$

$$Retentivity ( \% ) = retention / intake \times 100$$

When the chondroprotective nutraceutical composition is administered to a mammal in a therapeutically effective amount in combination with calcium, calcium absorptivity and calcium retentivity can increase by greater than about 15%, such as greater than about 20%, such as greater than about 25%, such as greater than about 30%, such as greater than about 35%, such as greater than about 40%, such as greater than about 45%, such as greater than about 50%, such as greater than about 55%, such as greater than about 60%, such as greater than about 65%, such as greater than about 70% in relation to the same mammal prior to treatment or to a mammal or a group of mammals that is not administered the chondroprotective nutraceutical composition.

Additionally, mammals administered the chondroprotective nutraceutical composition of the present disclosure may also exhibit a dramatic increase in femur dry weight as compared to the same mammal or a group of mammals that have not been administered the chondroprotective nutraceutical composition. For example, after 12 weeks of administration of the chondroprotective nutraceutical composition at a therapeutically effective amount, femur dry weight can increase by greater than about 10%, such as greater than about 20%, such as greater than about 30%, such as greater than about 40%, such as greater than about 50%, and generally less than about 80%, such as less than about 70%.

Various different bone markers and/or gene expression markers can also be influenced by the chondroprotective nutraceutical composition according to the present disclosure. For instance, administering the chondroprotective nutraceutical composition to a mammal in a therapeutically effective amount can (in relation to the same mammal prior to treatment or in relation to a mammal or group of mammals not administered the chondroprotective nutraceutical composition) increase RANK, which promotes bone formation, in an amount greater than about 50%, such as in an amount greater than about 80%, such as in an amount greater than about 100%, such as in an amount greater than about 120%, such as in an amount greater than about 150%, and generally in an amount less than about 250%. RANK-L, which is a marker for bone resorption, can decrease by at least about 10%, such as at least about 20%, such as at least about 30%, such as at least about 40%, such as at least about 50%. Osteoprotegerin, which protects the skeleton from excessive bone resorption, can increase by at least about 25%, such as by at least about 50%, such as by at least about 75%, such as by at least about 100%, such as at least by about 125%, and generally less than about 300%. SOX-9 is an osteo-marker related to the sex determining region Y. It was discovered that the chondroprotective nutraceutical composition of the present disclosure can significantly reduce SOX-9 levels in the mammal, such as by greater than about 15%, such as by greater than about 20%, such as by greater than about 25%, such as by greater than about 30%, such as by greater than about 40%, such as by greater than about 50%, such as even greater than about 55%.

The chondroprotective nutraceutical composition of the present disclosure can also have a dramatic influence on insulin-like growth factor 1 (IGF-1) and/or on bone morphogenetic protein 2 (BMP2) in the body of the mammal. For example, administering a therapeutically effective amount of the chondroprotective nutraceutical composition to a mammal over a period of time, such as 12 weeks, can increase IGF-1 by greater than 100%, such as by greater than about 150%, such as greater than about 200%, such as greater than about 250%, and generally less than about 500% in comparison to the same mammal that has not been administered the chondroprotective nutraceutical composition.

Administering a therapeutically effective amount of the chondroprotective nutraceutical composition to a mammal over a period of time, such as 12 weeks, can increase BMP2 by greater than at least about 30%, such as greater than at least about 50%, such as greater than at least about 75%, such as greater than at least about 100%, such as greater than at least about 125%, such as greater than at least about 150%, such as greater than at least about 175%, and generally less than about 300% in comparison to the same mammal that has not been administered the chondroprotective nutraceutical composition.

The chondroprotective nutraceutical composition may include one or more of any collagens and/or, in one aspect, may include one or more of Type I collagen, Type II collagen, Type III collagen, Type IV collagen, or collagen peptides, or a mixture thereof. In one aspect, the chondroprotective nutraceutical composition contains Type II collagen alone or in combination with one or more of Type I collagen, Type III collagen, Type IV collagen, or collagen peptides. In one aspect, the chondroprotective nutraceutical composition may include a mixture of Type II collagen (sometimes referred to as native Type II collagen) and undenatured Type II collagen. Additionally or alternatively, the chondroprotective nutraceutical composition may include a mixture of native Type II collagen and undenatured Type II collagen, in addition to a further collagen, such as Type I, Type III, Type IV, or collagen peptides. Furthermore, in one aspect, the chondroprotective nutraceutical composition includes whole collagen protein, biologically active peptide fragments of collagen, or a combination thereof.

In one aspect, one or more types of collagens in the chondroprotective nutraceutical composition may be heat sterilized, such as by autoclaving, and/or may also include salts, such as alkalizing or acid, and/or organic or inorganic salts. Thus, in one aspect, a portion of the collagen may be at least partially hydrolyzed. In one aspect, the collagen is hydrolyzed by any process or compound, including by an acid base agent, an enzyme, heat or other temperature extreme, a chemical, UV, a salt, or combinations thereof. In one aspect, the hydrolyzed collagen is undenatured collagen that has been at least partially hydrolyzed by any method, and in one aspect, may include undenatured collagen that has any portion that has been denatured. However, as discussed, in one aspect, at least a portion of the chondroprotective nutraceutical composition is undenatured.

Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II brand from InterHealth Nutraceuticals. UC-II® brand is a natural ingredient that contains a glycosylated, undenatured Type II collagen. The chondroprotective nutraceutical composition can also comprise a hydrolyzed collagen. The chondroprotective nutraceutical composition can also comprise a pure protein or active peptide fragments. In one embodiment, the chondroprotective nutraceutical composition can be free of any bone or bone material. In other embodiments, the chondroprotective nutraceutical composition can be free of any transforming growth factors (TGFs), bone morphogenetic proteins (BMPs), or both. In still another embodiment, the chondroprotective nutraceutical composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the Type II collagen in the tissue, such as low-temperature processing, and formed into doses containing therapeutically effective levels of undenatured Type II collagen. Being a natural product some variation from sample to sample is to be expected. These variations can be minimized by blending after comminution. The blending can be aided by analytical techniques which allow the measurement of the amount of undenatured Type II collagen and other constituents.

By forming the particles and sterilizing the Type II collagen as discussed above, the undenatured Type II collagen may be resistant to gastric acid and digestive enzymes in the stomach. Due to this sterilization process, the undenatured Type II collagen also retains its 3-dimensional shape, preserving the bioactive epitope regions. Without wishing to be bound by theory, it is believed that the epitope regions contain the ability to induce oral tolerance. Particularly epitope regions allow undenatured collagen to bind to the Peyer's Patches, which have the ability to induce oral tolerance processes.

The chondroprotective nutraceutical composition of the present disclosure can be administered to mammals orally in the form of individual dosage vessels. Each dose can contain a therapeutically effective amount of collagen based upon the type of mammal being treated, the condition of the mammal, and various other factors. In one aspect, each dose of the chondroprotective nutraceutical composition can contain collagen in an amount of at least about 10 mg, such as at least about 20 mg, such as at least about 40 mg such as at least about 60 mg, such as at least about 80 mg, such as at least about 100 mg, such as at least about 150 mg, such as at least about 200 mg, such as at least about 250 mg, such as at least about 300 mg, such as at least about 350 mg, such as at least about 400 mg, such as at least about 500 mg, such as at least about 600 mg, such as at least about 700 mg, such as at least about 800 mg, such as at least about 900 mg, such as at least about 1,000 mg, such as at least about 1,100 mg, such as at least about 1,200 mg, such as at least about 1,300 mg, such as at least about 1,400 mg, such as at least about 1,500 mg, such as at least about 1,600 mg, such as at least about 1,700, mg such as at least about 1,800 mg, such as at least about 1,900 mg, such as at least about 2,000 mg. In general, each dose of the chondroprotective nutraceutical composition can contain collagen in an amount less than about 5,000 mg, such as in an amount less than about 3,000 mg, such as in an amount less than about 2,000 mg, such as in an amount less than about 1,500 mg. Furthermore, it should be understood that, in one aspect, the chondroprotective nutraceutical composition may be a Type II chondroprotective nutraceutical composition, where substantially all of the collagen in the chondroprotective nutraceutical composition is Type II collagen.

In one aspect, the amount of the Type II chondroprotective nutraceutical composition present in the supplement is based upon the type of mammal and/or the body weight of the healthy mammal. For example, collagen can be present in each dose in an amount greater than about 0.2 mg/kg of body weight, such as greater than about 0.5 mg/kg of body weight, such as greater than about 1 mg/kg of body weight, such as greater than about 3 mg/kg of body weight, such as greater than about 5 mg/kg of body weight, and generally less than about 10 mg/kg of body weight, such as less than about 3 mg/kg of body weight.

In one aspect, undenatured Type II collagen may form all, or substantially all, of the total Type II collagen in the chondroprotective nutraceutical composition, and therefore, may be present in the supplement in the above discussed amounts. However, in one aspect, undenatured Type II collagen may account for about 0.5% to about 95% of the total Type II collagen and/or chondroprotective nutraceutical composition, such as about 1% to about 75%, such as about 1.5% to about 50%, such as about 2% to about 40%, such as about 2.5% to about 15% of the total Type II collagen or total chondroprotective nutraceutical composition, or any ranges or values therebetween.

In one aspect, the chondroprotective nutraceutical composition may further include a preservative salt, such as potassium chloride. Thus, in one aspect, the total amounts of chondroprotective nutraceutical composition discussed above may include Type II collagen and/or undenatured Type II collagen, alone or in combination with a further collagen, a preservative salt, or combinations thereof. In such as aspect, the total Type II collagen, including native and undenatured Type II collagen, may account for about 1% to about 99% of the chondroprotective nutraceutical composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween.

In one aspect, when the Type II collagen includes undenatured Type II collagen, the undenatured Type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a chondroprotective nutraceutical composition having an undenatured Type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a chondroprotective nutraceutical composition having an undenatured Type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a chondroprotective nutraceutical composition having an undenatured Type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, a chondroprotective nutraceutical composition having an undenatured Type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, a chondroprotective nutraceutical composition having an undenatured Type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, a chondroprotective nutraceutical composition having an undenatured Type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TF/g or greater, such as up to about 50 μmol TF/g or greater, up to about 75 μmol TF/g, or any ranges or values therebetween.

When the Type II collagen includes undenatured Type II collagen, the undenatured Type II collagen may have a molecular weight of about 10,000 Daltons or more, such as about 15,000 Daltons or more, such as about 20,000 Daltons or more, such as about 25,000 Daltons or more, such as about 30,000 Daltons or more, such as about 35,000 Daltons or more, such as about 40,000 Daltons or more, such as about 45,000 Daltons or more, such as about 50,000 Daltons or more, such as about 55,000 Daltons or more, such as about 60,000 Daltons or more, such as about 65,000 Daltons or more, such as about 70,000 Daltons or more, such as about 75,000 Daltons or more, such as about 80,000 Daltons or more, such as about 85,000 Daltons or more, such as about 90,000 Daltons or more such as about 95,000 Daltons or more, such as about 100,000 Daltons or more, up to about 350,000 Daltons or less, or any ranges or values therebetween.

While various aspects and benefits have been discussed, in one aspect, the chondroprotective nutraceutical composition is incorporated into a suitable delivery form prior to incorporation into a dosage form as discussed below. In one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the Type II collagen), and the emulsion may be used to incorporate the composition into a water-based application. However, it should be understood that, in one aspect, a water-soluble Type II collagen may be used in the supplement.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In some aspects, the oil-in-water emulsion may contain one or more stabilizers or suspension promoting agents. For example, in certain aspects, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, Vitamin E sourced from mixed tocopherols, beta carotene, ubiquinone, lecithin, sunflower lecithin, Vitamin D, cannabinoids, hemp extracts, Vitamin K, phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water-soluble phases, or the oil/fat soluble phase.

In some aspects, the oil-in-water emulsion disclosed herein may be used any suitable dosage form, such as tablets, gummy chewables, edible films, lozenges, liquid suspensions, syrups, lipid micelles, spray-dried dispersions, nanoparticles, and the like, which may also be incorporated into a further supplement.

Alternatively, the oil-in-water emulsion may be contained in a nutritional product, such as a supplement, a food product, or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

The nutritional product can include any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, powder, or the like

In one aspect, the supplement according to the present disclosure may be administered to the mammal including by oral, enteral or by-inhalation administration of whole collagen protein or biologically active peptide fragments of collagen. For instance, in one aspect, it is believed that the whole collagen protein or biologically active peptide fragments of collagen enhances the content of trans-L-hydroxyproline based on the total weight of amino acids contained in collagen Type II and enables efficient production of trans-L-hydroxyproline to enhance the efficacy.

The supplement composition of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, fats, oils and/or waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the supplement composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the supplement composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the supplement composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Consumer Health Inc, having offices in Morristown. NJ USA.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the supplement composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

As described above, the chondroprotective nutraceutical composition or supplement of the present disclosure can contain collagen in combination with one or more bone health additives. In one embodiment, for instance, the composition or supplement contains a calcium source. The calcium source, for example, may comprise calcium carbonate, calcium citrate, calcium gluconate, calcium lactate, calcium hydroxyapatite, or mixtures thereof. Calcium can be present in each dose in an amount greater than about 100 mg, such as greater than about 200 mg, such as greater than about 250 mg, such as greater than about 300 mg, such as greater than about 350 mg, such as greater than about 400 mg, such as greater than about 500 mg, such as greater than about 600 mg, such as greater than about 700 mg, such as greater than about 800 mg, such as greater than about 900 mg, such as greater than about 1,000 mg, such as greater than about 1,200 mg, such as greater than about 1,400 mg, such as greater than about 1,600 mg, such as greater than about 1,800 mg, such as greater than about 2,000 mg, such as greater than about 2,500 mg, and generally less than about 5,000 mg, such as less than about 3,000 mg, such as less than about 2,500 mg.

Other bone health additives that may be contained in the composition include Vitamin K2, Vitamin D3, an antiresorptive agent, an anabolic agent, or mixtures thereof. Each bone health additive can be present in the composition in relation to the collagen at a weight ratio of from about 5:1 to about 1:1,000. For instance, the bone health additive can be present in relation to the collagen at a weight ratio of from about 1:1 to about 1:500, such as from about 1:10 to about 1:300, such as from about 1:100 to about 1:200.

In addition to calcium, the composition can contain one or more other minerals, such as potassium, manganese, magnesium, zinc, or mixtures thereof. Minerals may be contained in the supplement in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the supplement contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the supplement contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the supplement contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

The supplement can be administered with or combined with various other health additives. For example, the chondroprotective nutraceutical composition can contain or be administered with antioxidants, anti-inflammatory agents, or combinations thereof.

The supplement may be suitable for administration to any mammal. For instance, the mammal may be human, canine, feline, bovine, equestrian or the like. The composition can be fed to a mammal of any age such as from parturition through the adult life in the mammal. In various embodiments the mammal may be a human, dog, a cat, a horse, a pig, a sheep, or a cow.

The present disclosure may be better understood according to the following example, which is intended to be non-limiting and exemplary in nature.

### Example 1

### Material and Methods

Weaned rats about 4 weeks old, weighing about 60-80 grams of the same sex, 10 rats in each group were used. Animals were reared at the temperature of (22±2° C.), humidity (55 f 5%) and a 12/12 h light/dark cycle. Weaned rats born 4 weeks after the adaptation period for one week, were fasted for 12 hours, weighed, grouped randomly according to body weight, and reared in separate cages. They were fed deionized water to avoid getting calcium from drinking water.

The following feed formula was used during the study:

TABLE 1
Basic feed formula (adjust Ca2+ to 150 mg/100 g feed)¹

| Ingredient | g/kg diet |
|---|---|
| Casein | 200.000 |
| Cornstarch | 397.486 |
| Dextrin | 132.000 |
| Sucrose | 100.000 |
| Vegetable oil | 70.000 |
| Fiber | 50.000 |
| Mineral mix * | 35.000 |
| Vitamin mix ** | 10.000 |
| L-Cystine | 3.000 |
| Choline bitartrate | 2.500 |
| Tert-butylhydroquinone | 0.014 |

¹Values were taken from Reeves et al. (1993a).
* Mineral mix (g/kg mix): Calcium carbonate, anhydrous, (40.04% Ca) 36.00; Potassium phosphate, monobasic (22.76% P, 28.73% K) 196.00; Potassium citrate, tri-potassium, monohydrate, (36.16% K) 70.78; Sodium chloride, (39.34% Na, 60.66% CI) 74.00; Potassium sulfate, (44.87% K, 18.39% S) 46.60; Magnesium oxide, (60.32% Mg) 24.00; Ferric citrate (52.14% Fe) 6.06; Zinc carbonate, (52.14% Zn) 1.65; Manganous carbonate, (47.79% Mn) 0.63; Cupric carbonate (57.47% Cu) 0.30; Potassium iodate, (59.3% I) 0.01; Sodium selenate, anhydrous, (41.79% Se) 0.01025.
** Vitamin mix (g/kg): Nicotinic acid 3.0; Pantothenic acid 1.5; Pyridoxine-HCI 0.7; Thiamin-HCI 0.6; Riboflavin 0.60; Folic acid 0.20; D-Biotin 0.02; Vitamin B-12 (cyanocobalamin) (0.1% in mannitol) 2.50; Vitamin E (all-rac-α-tocopheryl acetate) (500 IU/g) 15.00; Vitamin A (all-trans-retinyl palmitate) (500,000 IU/g) 0.80; Vitamin D₃ (cholecalciferol) (400,000 IU/g) 0.25; Vitamin K (phylloquinone) 0.075.

### Experimental Design

To assess the undenatured form of Type II collagen (UCII) efficacy on Ca absorption bone quality, 4-week-old male Wistar rats were fed a normal diet for a 1-week adaptation period before the study.

The rats were randomly assigned to six diet groups as follows:
1) Negative control (0.150% Ca)
2) Negative control (0.150% Ca)+0.66 mg/kg BW UCH (40 mg HED based on 60 kg BW)
3) Negative control (0.150% Ca)+1.32 mg/kg BW UCII (80 mg HED based on 60 kg BW)
4) Negative control (0.150% Ca)+3.3 mg/kg BW UCH (200 mg HED based on 60 kg BW)
5) Negative control (0.150% Ca)+6.6 mg/kg BW UCII (400 mg HED based on 60 kg BW)
6) Negative control (0.150% Ca)+19.8 mg/kg BW UCH (1200 mg HED based on 60 kg BW)

The test substance was given by oral gavage, and the test substance was mixed into the feed when the gavage failed. Weight of each rat was determined weekly. After 12 weeks, blood, liver, kidney and bone (right femur weights were measured while left femurs were excised for the measurement of BMD) samples were collected.

### Metabolism Experiment

At the last week of the experiment, a calcium metabolism experiment was carried out for 3 days. The food intake for 3 days was recorded and 72-hour feces were collected to determine the calcium content in the feed and feces.

### Determination of Calcium in Blood, Feed and Feces: Determination by Atomic Absorption Spectrophotometry

The feed samples were evenly mixed and passed through a 20-mesh sieve; the rat feces samples were dried in an oven at 60° C., cooled in a desiccator, and ground for mineral analyses. Approximately 0.3 g of ground samples was mineralized in HNO₃ (5 ml) using a closed microwave digestion system (Berghof, Eningen, Germany). The feed and feces samples were then diluted with deionized water, and the Ca content in feces and diet was determined. Lanthanum chloride (Merck, Darmstadt, Germany) was added as an interference suppressant for Ca g analyses. Ca levels in all samples were measured by atomic absorption spectrometry (AAS, Perkin-Elmer, Analyst 800, Norwalk, CT, USA) with flame atomization in an acetylene-air at the 422.7 nm wavelength, with the Zeeman background correction. The assessments were performed in triplicate. Serum Ca levels also were measured by atomic absorption spectrometry Calculation

$$Intake of calcium ( mg / d ) = calcium content in feed ( mg / g ) \times feed consumption ( g / d )$$

$$Fecal calcium ( mg / d ) = Fecal calcium content ( mg / g ) \times Fecal excretion ( g / d )$$

$$Apparent calcium absorption rate ( \% ) = ( calcium intake - calcium in feces ) / calcium intake \times 100 \%$$

### Femur Weight Measurement

The animals were sacrificed after being fed for 3 months. The right femur was stripped and baked in an oven at 105° C. to a constant weight, and the bone-dry weight was measured.

### Determination of Femoral Bone Mineral Density

Bone mineral density was measured by dual-energy X-ray bone densitometer at the midpoint of the femur and the distal end of the femur. Marks were drawn on the distal end of the femur and the midpoint of the femur to determine the measurement point. The full length of the femur was measured and a straight line was drawn along the cross-sectional direction from its midpoint. The measurement point of the distal end of the femur was determined at the lower edge of the joint groove of the distal end of the femur and a straight line parallel to the mark was drawn at the midpoint of the femur through this point. This is the measurement point of the distal end of the femur.

The bone to be measured was moved so that the marker line of the measured point coincides with the vertical projection of the moving path of the probe on the measuring table. The measurement was started and repeated twice for each point. If the two results were not parallel (the error is greater than 10%) the measurement was repeated. The two results were averaged, BMC (bone mineral content), BW (bone width) and the result is BMD (bone mineral density) were determined.

### Determination of Bone Calcium Content and Calcium in Feed Material

Bone calcium content and feed calcium content was measured by an Atomic Absorption Spectrometry (AAS) method. Approximately 0.3 g of ground samples was mineralized in HNO₃ (5 ml) using a closed microwave digestion system (Berghof, Eningen, Germany). The samples were then diluted with deionized water, and the Ca content in tissue and diet was determined. Lanthanum chloride (Merck, Darmstadt, Germany) was added as an interference suppressant for Ca g analyses. Ca levels in all samples were measured by atomic absorption spectrometry (AAS, Perkin-Elmer, Analyst 800, Norwalk, CT, USA) with flame atomization in an acetylene-air at the 422.7 nm wavelength, with the Zeeman background correction. The assessments were performed in triplicate.

Osteocalcin levels in sera was detected with an ELISA (Elx-800, Bio-Tek Instruments Inc, Vermont, USA) device using commercial kits specific to rats according to manufacturer's instructions.

OPG, RANK, RANKL, SOX-9, BMP2, IGF-1 was measured by WB.

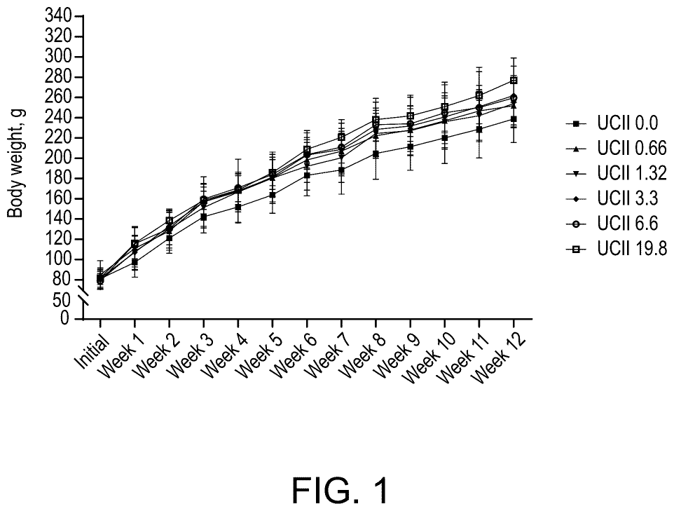

The results of body weight measurements are indicated below and shown in FIG. 1.

| | Groups | | | | | |
|---|---|---|---|---|---|---|
| | UCII 0.0 | UCII 0.66 | UCII 1.32 | UCII 3.3 | UCII 6.6 | UCII 19.8 |
| Initial | 81.00 | 84.90 | 79.30 | 81.50 | 78.80 | 81.70 |
| Week 1 | 97.10 | 111.00 | 107.80 | 107.10 | 115.50 | 116.00 |
| Week 2 | 121.10 | 128.10 | 130.00 | 133.00 | 130.40 | 138.60 |
| Week 3 | 142.20 | 157.20 | 151.30 | 157.00 | 159.60 | 158.50 |
| Week 4 | 151.90 | 167.90 | 166.70 | 167.00 | 170.80 | 168.70 |
| Week 5 | 163.80 | 180.70 | 180.40 | 181.40 | 184.00 | 185.90 |
| Week 6 | 183.10 | 198.30 | 192.10 | 203.10 | 203.60 | 208.80 |
| Week 7 | 188.40^{b} | 207.00^{ab} | 200.40^{ab} | 208.70^{ab} | 211.00^{ab} | 220.70^{a} |
| Week 8 | 204.70^{b} | 222.20^{ab} | 224.50^{ab} | 228.40^{ab} | 233.10^{ab} | 238.10^{a} |
| Week 9 | 211.40 | 227.80 | 227.20 | 231.80 | 234.10 | 241.80 |
| Week 10 | 220.00 | 236.90 | 236.10 | 240.80 | 244.90 | 250.90 |
| Week 11 | 228.70 | 246.70 | 242.00 | 250.90 | 250.00 | 261.90 |
| Week 12 | 238.70^{b} | 251.80^{ab} | 254.00^{ab} | 261.90^{ab} | 259.60^{ab} | 276.90^{a} |

Calcium absorption and retention results are as follows:

| | Groups | | | | | |
|---|---|---|---|---|---|---|
| | UCII 0.0 | UCII 0.66 | UCII 1.32 | UCII 3.3 | UCII 6.6 | UCII 19.8 |
| Intake Ca mg/day | 32.60 | 33.37 | 33.37 | 33.88 | 32.85 | 34.65 |
| Feces Ca mg/day | 25.37^{a} | 24.45^{a} | 23.34^{ab} | 22.66^{ab} | 20.68^{b} | 21.15^{b} |
| Urine Ca mg/day | 0.54 | 0.53 | 0.50 | 0.51 | 0.49 | 0.50 |
| Absorptivity,% | 22.19^{e} | 26.76^{d} | 30.09^{c} | 33.09^{b} | 37.05^{a} | 38.99^{a} |
| Retentivity, % | 20.54^{e} | 25.16^{d} | 28.58^{c} | 31.59^{b} | 35.55^{a} | 37.55^{a} |

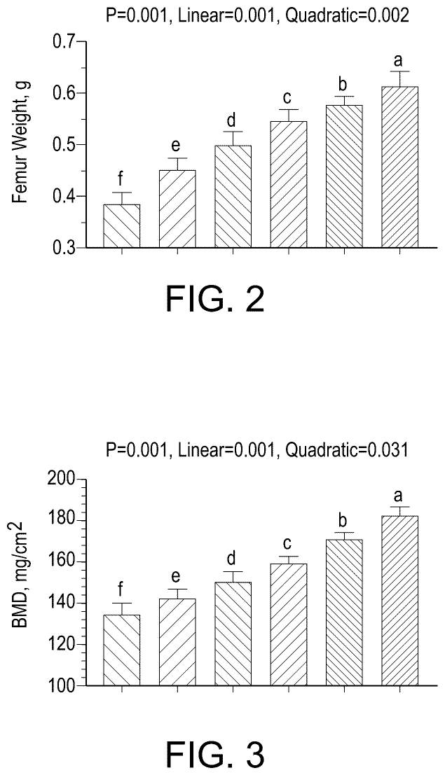

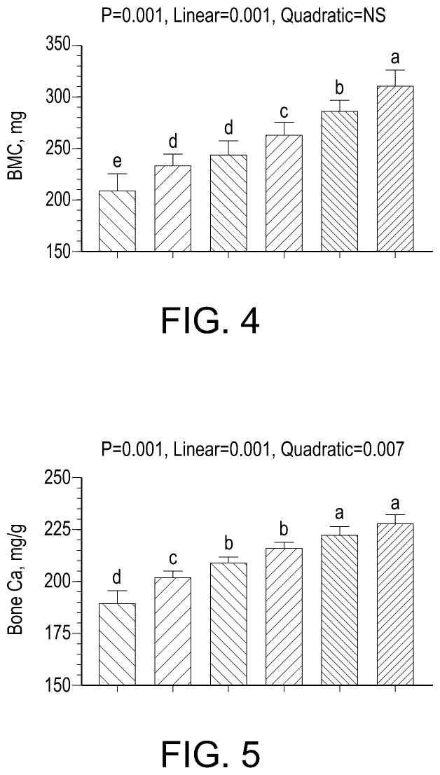

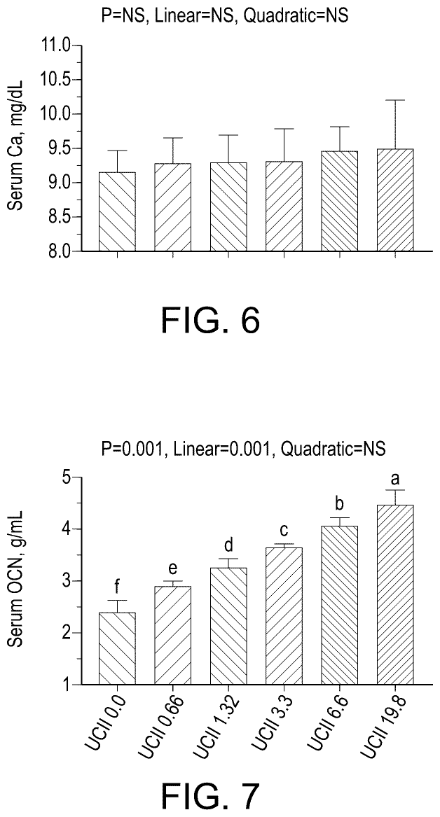

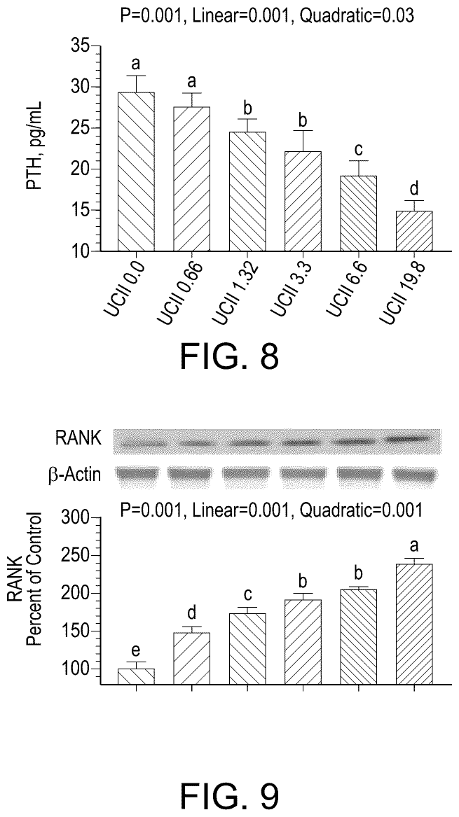

The effect of different doses of undenatured collagen Type II on femur dry weight, bone mineral density, bone mineral content, bone calcium, serum calcium, osteocalcin, and parathyroid hormone levels in rats during the study are presented below after 12 weeks. FIGS. 2-8 also illustrate the results.

| | Groups | | | | | |
|---|---|---|---|---|---|---|
| | UCII 0.0 | UCII 0.66 | UCII 1.32 | UCII 3.3 | UCII 6.6 | UCII 19.8 |
| Femur Weight, g | 0.39^{f} | 0.45^{e} | 0.50^{d} | 0.55^{c} | 0.58^{b} | 0.61^{a} |
| BMD, mg/cm² | 134.19^{f} | 142.3^{e} | 150.26^{d} | 159.41^{c} | 170.99^{b} | 182.13^{a} |
| BMC, mg | 209.78^{e} | 234.39^{d} | 244.58^{d} | 262.84^{c} | 287.03^{b} | 310.85^{a} |
| Bone Ca, mg/g | 189.24^{d} | 201.83^{c} | 209.60^{b} | 215.96^{b} | 222.74^{a} | 228.22^{a} |
| Serum Ca, mg/dL | 9.16 | 9.29 | 9.30 | 9.32 | 9.47 | 9.49 |
| OCN, ng/mL | 2.40^{f} | 2.89^{a} | 3.26^{d} | 3.64^{c} | 4.05^{b} | 4.47^{a} |
| PTH, pg/mL | 29.34^{d} | 27.69^{c} | 24.69^{b} | 22.30^{b} | 19.24^{a} | 14.8^{a} |

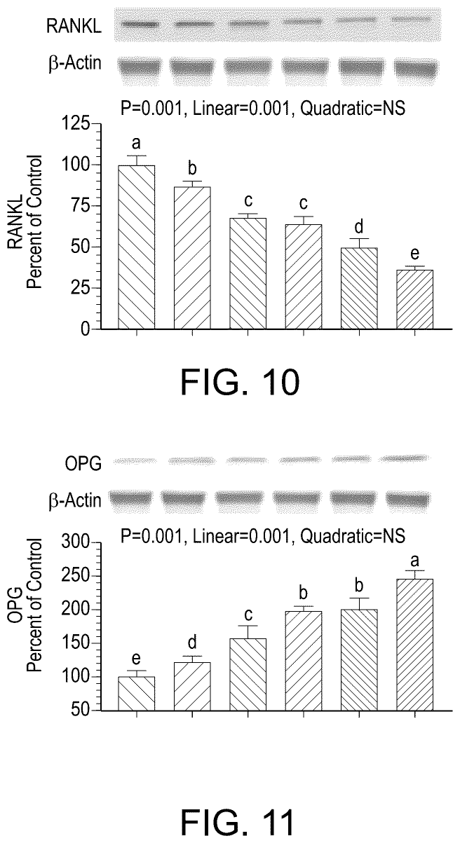

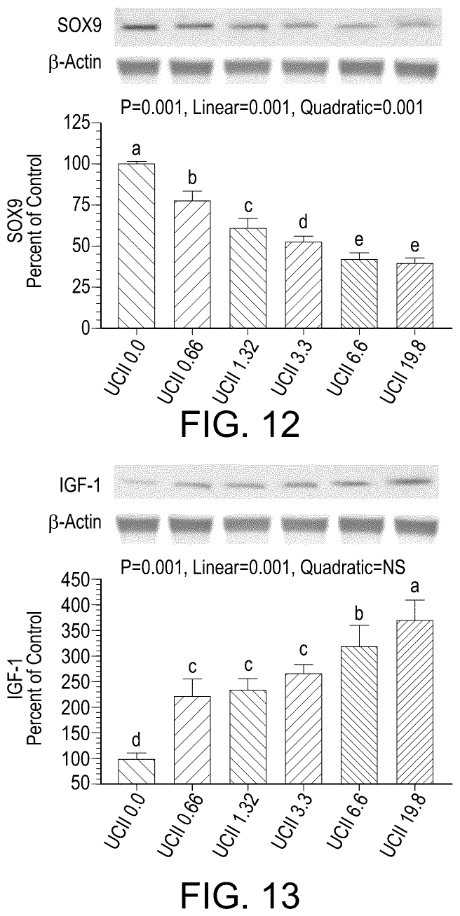

The effect of different doses of undenatured collagen Type II (UCII) on levels of receptor activator of nuclear factor κB (RANK, Panel A), receptor activator of nuclear factor κB ligand (RANKL, Panel B), osteoprotegerin (OPG, Panel C), sex-determining region Y-box 9 (SOX-9, Panel D, and RANKL/OPG ratio (Panel E) in bone in rats fed with low Ca (0.15%) containing diet for 12 weeks are illustrated in FIGS. 9-12.

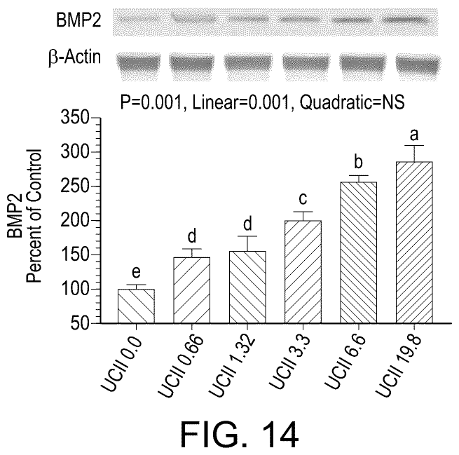

The effect of different doses of undenatured collagen Type II (UCII) on levels of insulin-like growth factor 1 (IGF-1, Panel A), and bone morphogenetic protein 2 (BMP2, Panel B) proteins in bone in rats fed with low Ca (0.15%) containing diet for 12 weeks are illustrated in FIGS. 13 and 14.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method for increasing calcium absorption in a mammal, the mammal having a diet that includes calcium, the method comprising:
administering to the mammal a therapeutically effective amount of a chondroprotective nutraceutical composition sufficient to increase calcium absorption in the mammal, the chondroprotective nutraceutical composition comprising a collagen.

2. A method as defined in claim 1, wherein the mammal has a calcium deficiency.

3. A method as defined in claim 1, wherein the mammal is:
(i) a human,
(ii) healthy,
(iii) suffering from a joint injury,
(iv) suffing from a skeletal disease, or
(vi) a young healthy adult who is at the growth phase of bone development.

4. (canceled)

5. A method as defined in claim 1, wherein the method is for treating a bone disease, a joint disease, or a co-morbidity, including diabetes, cardiometabolic syndrome, an inflammatory and immune disease condition or any chronic disease condition related thereto, or wherein the method is to meet a nutritional need or requirement for a therapeutic or pharmaceutical purpose in the mammal.

6. (canceled)

7. A method as defined in claim 1, wherein the chondroprotective nutraceutical composition is administered to the mammal in an amount sufficient to increase:
(i) bone mineral density in the mammal, wherein the increase in hone mineral density is greater than about 4%; in comparison to the mammal prior to administration of the chondroprotective nutraceutical composition to the mammal,
(ii) percent absorptivity of calcium in the mammal, wherein the increase in percent absorptivity of calcium is greater than about 1% in comparison to the mammal prior to
(ii) percent retentivity of calcium in the mammal, wherein the percent retentivity of calcium is increased by greater than about 1S % in comparison to the mammal prior to administration of the chondroprotective nutraceutical composition to the mammal,
(iv) dry femur weight in the mammal,
(v) osteocalcin in the mammal, wherein the increase in osteocalcin is greater than about 10%, in comparison to the mammal prior to administration of the chondroprotective nutraceutical composition to the mammal, or
(vi) bone morphogenetic protein 2 in the mammal, wherein the increase in bone morphogenetic protein 2 is greater than about 30% in comparison to the mammal prior to administration of the chondroprotective nutraceutical composition to the mammal.

8. (canceled)

9. (canceled)

10. (canceled)

11. (canceled)

12. (canceled)

13. (canceled)

14. A method as defined in claim 1, wherein the collagen is obtained from chicken, fish, bovine or porcine cartilage.

15. (canceled)

16. A method as defined in claim 1, wherein the chondroprotective nutraceutical composition is administered to the mammal at least once a week or wherein the chondroprotective nutraceutical composition is administered to the mammal based on the amount of calcium that is in a blood of the mammal.

17. (canceled)

18. A method as defined in claim 1, wherein the collagen comprises a Type I collagen, a Type II collagen, a Type III collagen, a Type IV collagen, or a Type V collagen.

19. A method as defined in claim 1, wherein the chondroprotective nutraceutical composition is administered to the mammal at collagen dose levels of at least about 10 mg of collagen per dose and less than about 3,000 mg of collagen per dose.

20. A method as defined in claim 1, wherein the chondroprotective nutraceutical composition is administered to the mammal in combination with a calcium supplement.

21. A method as defined in claim 20, wherein the calcium supplement is contained within the chondroprotective nutraceutical composition.

22. A method as defined in claim 1, wherein the chondroprotective nutraceutical composition contains an antioxidant, an anti-inflammatory agent, or mixtures thereof.

23. A method as defined in claim 1, wherein the diet of the mammal is calcium deficient.

24. A chondroprotective supplement for improving bone health in a mammal comprising:
a bone health additive; and
a bone health adjuvant for the bone health additive, the bone health adjuvant comprising a collagen, the collagen being present in the chondroprotective supplement in a therapeutically effective amount sufficient to increase absorption of the bone health additive into a bone of a mammal.

25. A chondroprotective supplement as defined in claim 24, wherein the bone health additive comprises calcium, and wherein calcium is present in the supplement in an amount from about 250 mg to about 2,500 mg.

26. A chondroprotective supplement as defined in claim 25, wherein the bone health additive comprises calcium carbonate, calcium citrate, calcium gluconate, calcium lactate, or mixtures thereof.

27. A chondroprotective supplement as defined in claim 24, wherein the collagen is present in the supplement in an amount greater than about 10 mg and in an amount less than about 500 mg.

28. A chondroprotective supplement as defined in claim 24, wherein the bone health adjuvant is present in the supplement in an amount sufficient to increase osteocalcin in the mammal and/or bone morphogenetic protein 2 in the mammal.

29. A chondroprotective supplement as defined in claim 24, wherein the supplement is in the form of a tablet, a capsule, a suspension, or an emulsion.

30. A chondroprotective supplement as defined in claim 24, wherein the bone health adjuvant is present in the supplement in an amount sufficient to increase percent absorptivity or percent retentivity of the bone health additive in the mammal.
